(19) United States
(12) Patent Application Publication
(10) Pub. No.: US 2017/0198002 A1
(43) Pub. Date: Jul. 13, 2017

(54) High Purity Low Endotoxin Carbohydrate (HPLE) Compositions, and Methods of Isolation Thereof

(71) Applicant: Aventor Performance Materials, Inc., Center Valley, PA (US)

(72) Inventors: Bhaktavachalam THIYAGARAJAN, Bethlehem, PA (US); Martina COX, Allentown, PA (US); Nandu DEORKAR, Cedar Knolls, NJ (US)

(73) Assignee: Avantor Performance Materials, Inc., Center Valley, PA (US)

(21) Appl. No.: 15/316,614

(22) PCT Filed: Dec. 23, 2014
(86) PCT No.: PCT/US14/72117

§ 371 Date: Dec. 6, 2016

Related U.S. Application Data
(60) Provisional application No. 62/011,810, filed on Jun. 13, 2014.

(51) Int. Cl. C07H 1/06; C07H 3/02; C07H 3/04

(52) U.S. Cl. CPC C07H 1/06; C07H 3/04; C07H 3/02

(57) ABSTRACT

Provided herein is a highly pure carbohydrate composition, and a method of making a highly pure carbohydrate composition. The method includes passing an aqueous carbohydrate solution through an anion exchange chromatography column including a polyethyleneimine (PEI) chromatographic media to obtain a purified solution, and isolating a highly pure carbohydrate composition from the purified solution.

### FIELD OF THE INVENTION

The present invention relates to high purity low endotoxin carbohydrates, and methods of making and using thereof.

### BACKGROUND

Carbohydrates or sugars are useful as formulation enhancers for various active agents, such as in an injectable formulation involving an active agent such as a protein or peptide. Additionally, carbohydrates may be used as cell culture or fermentation supplement. Carbohydrates including mono, di, tri and polysaccharides such as glucose, sucrose, galactose, trehalose, maltose, amylose, maltohexaose, maltoheptaose, maltotetraose have been found to be particularly useful in these applications.

As the case with most plant-derived natural substances, carbohydrates and sugars do not exist in nature in a naturally purified state. Table sugar (sucrose) for example, comes from plant sources, and is required to be extracted and purified therefrom. Two important sugar crops predominate: sugarcane (Saccharum spp.) and sugar beets (Beta vulgaris), in which sugar can account for 12% to 20% of the plants dry weight. Sucrose is typically obtained by extraction of these crops with hot water; concentration of the extract gives syrups, from which solid sucrose can be crystallized.

There are a number of notable differences between sugar and carbohydrates in their natural state, and after refinement and purification. Most notably, crystallization of the sugar is one major transformation. Still further however, even after refining and facile purification, sugars have a number of impurities inherently associated therewith. Such impurities, which will be discussed in more detail later in the application, include bacteria, protein, endotoxins, and various other plant-derived material.

Carbohydrates may be purified through many techniques, including by chromatographic separation. This can be done quickly and efficiently for laboratory scale synthesis, however, column chromatography and similar separation techniques become less useful as larger amounts of sugar are purified. The size of the column, amount of solvents and stationary phase (e.g. silica gel) required and time needed for separation each increase with the amount of product purified, making purification from multi-kilogram scale synthesis unrealistic using column chromatography.

Another common purification technique for sugars involves the use of an ion-exchange resin. This technique can be tedious, requiring a tedious pre-treatment of the ion exchange resin. Many available ion exchange resins are also not necessarily able to separate the sugars from salts (e.g., NaCl). Acidic resins tend to remove both metal ions found in the crude product and amino- or imino-sugars from the solution and are therefore not useful. After purification of a sugar using an ion exchange resin, an additional step of concentrating the diluted aqueous solution is often required, and may be problematic as this step can cause decomposition of the sugar, which produces contaminants, and also reduces the yield.

Similarly, other industrial and pharmaceutically useful sugars are commonly purified using chromatography and ion exchange resins that cannot easily be scaled up to the purification of multi-kilogram quantities. It is particularly important to remove impurities such as endotoxins from carbohydrates.

In chromatographic separatory techniques in general, a specific ligand is covalently attached to a solid support matrix. A sample containing the biological molecule which will specifically bind (absorb) to the immobilized ligand is brought into contact with the immobilized ligand. After unabsorbed and contaminating molecules are removed, the specifically bound molecule is eluted from the solid support by disrupting the specifically bound molecule-ligand interaction by one of several procedures, such as by changing the ionic strength or pH of elution buffers.

By this procedure, immobilized drugs, vitamins, peptides, hormones and the like may be used to isolate corresponding receptors or transport proteins. Immobilized protein can serve to isolate other complementary or interacting proteins. Similarly, such a procedure can be used to separate particulate biological specimens, such as cell membranes and even intact cells bearing specific receptors. Use of such a procedure is also useful to purify polynucleotides, antigens, antibodies, virus, enzymes and the like. In addition, such solid based affinity support matrixes have been utilized to immobilize enzymes for use in reactions as catalysts and the like.

Ion-exchange chromatography is a type of affinity chromatography where ions and/or polar molecules in a composition facilitate separation based on their affinity to the ion exchanger. Fine particles having an ion exchanging group are widely used as a separating material in the field of pure water production and chromatography. An anion exchanger having introduced therein polyethyleneimine as an ion exchanging group is used in the field of chelate resins, liquid chromatography for analyzing or isolating, for example, amino acids, peptide, protein, nucleic acids and saccharides.

Variety of anion exchange resins are available from various sources. They are prepared by attaching ligand to the solid support such as silica, Agarose or synthetic polymer. The anion exchange resins based on polyethylenimine is made by attaching polyethylenimine to a synthetic polymer or silica.

As examples of the method of making an anion exchanger comprised of a fine particle having introduced therein polyethyleneimine, there can be mentioned a method of introducing polyethyleneimine to a fine particle of a polymer having a halogenated alkyl group such as polychromethylstyrene as disclosed in U.S. Pat. No. 4,191,814; a method of introducing polyethyleneimine to an acrylate or methacrylate polymer having an epoxy group or a halogenated alkyl group as disclosed in U.S. Pat. No. 4,111,859; and a method of allowing an inorganic fine particle to adsorb polyethyleneimine and then crosslinking the adsorbed polyethyleneimine as disclosed in U.S. Pat. No. 4,245,005.

Endotoxins are small, stable, bacterially-derived hydrophobic molecules which can easily contaminate labware and whose presence can significantly impact both in vitro and in vivo experiments. Their presence is detected by the limulus amebocyte lysate (LAL) assay which can detect down to 0.01 Endotoxin Units (EU)/ml. The properties of high purity and low-endotoxin are needed when even the lowest level of contaminants, especially endotoxins (cell wall fragments of gram negative bacteria) and other high molecular weight impurities can compromise the final product's purity, biological activity, shelf-life or patient safety.

For carbohydrates used in pharmaceutical formulations or as a cell culture fermentation supplement, it is also critical to purify the carbohydrate such that is substantially free of endotoxins and other biological impurities such as DNA and RNA, heavy metals, related carbohydrate species, and bacterial contamination such as Ecoli.

An adequate process to safely remove endotoxins and other impurities to provide highly pure low endotoxin carbohydrates is therefore highly desirable.

### SUMMARY OF THE INVENTION

Provided therefore herein is a method of making a highly pure carbohydrate composition, and the highly pure composition resulting therefrom. The method includes passing an aqueous carbohydrate solution through an anion exchange chromatography column including a polyethylenimine (PEI) chromatographic media to obtain a purified solution, and isolating a highly pure carbohydrate composition from the purified solution. In an embodiment, the isolating step includes at least one of the steps of: i) crystallization with an alcohol, or ii) spray drying the purified solution.

In an embodiment, the alcohol used in the crystallization step is ethanol. In an embodiment, the method further includes a filtration step of the aqueous carbohydrate solution before step passing it through the PEI column. In an embodiment, the filter has a pore size of about 0.4 microns to about 0.5 microns.

In an embodiment, the highly pure carbohydrate composition is one selected from the group of sucrose, galactose, and trehalose. In an embodiment, the highly pure carbohydrate composition has endotoxin levels of less than 1 Endotoxin Unit per gram. In an embodiment, the highly pure carbohydrate composition has less than 5 ppb of elemental impurities such as lead. In another embodiment, the highly pure carbohydrate composition has less than 100 ppm of related carbohydrate species preferably less than 10 ppm.

In another embodiment, a highly pure carbohydrate composition made by the methods disclosed herein is provided. The composition comprises an aqueous carbohydrate solution having an endotoxin value of less than 1 Endotoxin Units per gram. In an embodiment, the aqueous carbohydrate solution has an endotoxin value of less than 0.4 Endotoxin Units per gram. In another embodiment, the aqueous carbohydrate solution has an endotoxin value of less than 0.3 Endotoxin Units per gram, and in another embodiment a value of about 0.1 Endotoxin Units per gram.

In an embodiment, the aqueous carbohydrate solution has been passed through an anion exchange chromatography column including a polyethylenimine (PEI) chromatographic media. In another embodiment, the aqueous carbohydrate solution if further isolated after passing through the column by at least one of the steps of: i) crystallization with an alcohol, or ii) spray drying said purified solution. In an embodiment, the highly pure carbohydrate composition has less than 5 ppb of elemental impurities such as lead.

In another embodiment, a formulation ingredient for a pharmaceutical composition is provided herein, particularly for a pharmaceutical formulation including a biologic. The formulation ingredient is a highly pure carbohydrate composition as described herein.

### DETAILED DESCRIPTION

The invention relates to composition and method to produce high purity low endotoxin (HPLE) carbohydrates such as Sucrose, Galactose, and Trehalose. In a preferred embodiment high purity low Endotoxin carbohydrates are the highly purified carbohydrates having very low levels of Endotoxin (less than 1 EU/g), a very low level of elemental impurities such as lead (<5 ppb), very low level of related carbohydrates species (less than 100 ppm), absence of bacterial contamination such as Ecoli and absence of RNA and DNA with no colored plant derived impurities. In a preferred composition, the endotoxin level is 0.6 EU/g and most preferred composition with the endotoxin level of less than 0.1 EU/g. The high purity low Endotoxin composition of carbohydrate is prepared by anion exchange chromatographic process followed by isolation using either (i) crystallization with ethanol or (ii) spray drying the purified sugar solution. In particular, Polymeric Polyethyleneimine (PEI) chromatographic media has been used to remove the contaminants such as Endotoxin and other biological impurities from aqueous sugar solution. Crystalline sugar from the purified sugar solution is isolated either by adding alcohol to a concentrated sugar solution or spray drying the purified sugar solution.

The purpose of the invention is to show that high purity Endotoxin free sugars can be obtained using anion-exchange chromatographic media especially using polymeric chromatographic media containing polyethyleneimine. The purified sugar solution can be isolated either by crystallization or by spray drying. The HPLE carbohydrates with the composition mentioned above may be used in many applications, including without limitation: the formulation of injectable drug such as protein, peptides or similar chemical entities, or used as cell culture and fermentation supplement.

The subject invention concerns the use of polymeric anion exchange resin, preferably polyethyleneimine chromatographic resin for the purification of sucrose, galactose and trehaolse dihydrate. In accordance with the present invention, raw sugars was dissolved in DI water and passed onto the chromatography column packed with anion exchange reins such as Poly PEI resin at flow rate of 100-500 cm/hour, with a concentration range of 100-500 mg/ml. Endotoxin and other anionic including biological impurities such as DNA and RNA being negatively charged at neutral pH strongly adsorbed to the column and purified sugar solution is collected. The material collected was concentrated under heat using vacuum and ethanol was added and kept in ice for few hours. It has been unexpectedly that the following factors play key role in successful crystallization: 1. concentration range of carbohydrates. The concentration range of the carbohydrates varied from 500-800 mg/ml. The preferred concentration range for sucrose is between 750-800 mg/ml, galactose is between 600-700 mg/ml and the trehalose dihydrate between 600-700 mg/ml. 2. The temperature of the concentrated solution before adding the alcohol. The temperature of the concentrated solution before adding alcohol was between 10-60° C. However, preferred temperature range is 24-60° C. and most preferred temperature is 40° C. 3. The amount of alcohol added if the alcohol was added at low temperature, it forms hard candy like material that sticks to the glasswares. The volume of alcohol added ranges from 2.5× to 3.0× to that of volume of concentrated solution and preferably 3.0×. The crystallized material was isolated by filtration and washed with ethanol and dried under vacuum. Alternatively, the purified solution can also be spray dried for isolation.

It is a surprising benefit of the present invention to obtain such purity levels in carbohydrate compositions. Other well-known methods of purifying carbohydrates, such as direct crystallization without chromatography purification, and hollow fiber filter cannot yield carbohydrate compositions of such purity. As a comparative example, the known purification technique of this type of crystallization yields a carbohydrate composition with much higher endotoxin levels, such as around 10 Eu/g, and contains other trace impurities such as RNA, DNA and other anionic impurities. As such, it is only possible to obtain such high purity levels by the present inventive process.

The carbohydrate compositions of the present invention are particularly useful in pharmaceutical compositions, such as parenteral compositions, including pharmaceutical compositions administered by methods other than enteral and topical administration, including by injection, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

It is very important, in particular for pharmaceutical compositions including a biologic as an active ingredient to have carbohydrate compositions of high purity. This is important because these carbohydrates are used for protein formulations that are administered through direct injection (parenteral formulation). The presence of even small amount of Endotoxin and other impurities will compromise the product purity, biological safety, shelf-life and patient safety.

There are a number of notable differences between sugar and carbohydrates in their natural state, and carbohydrates and sugar after isolation, refinement, and purification. Most notably, crystallization of the sugar is one major transformation. Still further however, even after refining and facile purification, sugars have a number of impurities inherently associated therewith. Such impurities include without limitation bacteria, various proteins, endotoxins, and various other plant-derived material. Typically the impurities exist in a mixture with the carbohydrates, and still remain with carbohydrates through the extraction process because of various ionic forces, and other bonding forces between the impurities and the carbohydrate. As such, the highly purified carbohydrates resulting from the present inventive process provides a novel composition of matter not existent in nature.

### EXAMPLES

The present invention is further exemplified, but not limited, by the following representative examples, which are intended to illustrate the invention and are not to be construed as being limitations thereto.

### Example 1

300 grams of cane Sucrose was dissolved in 800 mL of distilled water (DI)and diluted with additional DI water to 1 Liter (L). The sugar solution was filtered through a 0.45 micron filter and the solution was passed through a freshly packed PEI column (25.0×1.0 cm) at 4 mL/min. The solution was analyzed for Endotoxin. The endotoxin value was decreased from 7.4 Endotoxin Units per gram (EU/g) to<0.1 EU/g.

### Example 2

450 grams of beet Sucrose was dissolved in 800 mL of distilled (DI) water and diluted with additional DI water to 1 Liter (L). The sugar solution was filtered through a 0.45 micron filter and the solution was passed through a freshly packed PEI column (25.0×1.0cm) at 4 mL/min. The solution was analyzed for Endotoxin. The endotoxin value was decreased from 7.0 Endotoxin Units per gram (EU/g) to<0.1 EU/g. The material was free flowing with the mean particle size of 348 micron.

### Example 3

300 grams of trehalose dihydrate was dissolved in 800 ml DI water and diluted with additional DI water to 1L. The sugar solution was filtered through 0.45 micron filter and the solution was passed through the freshly packed PEI column (25.0×1.0 cm) at 4.5 ml/min. The solution was analyzed for Endotoxin. The Endotoxin value was decreased from 19 EU/g to 0.1 EU/g.

### Example 4

300 grams of Galactose was dissolved in 800 mL of distilled (DI) water and diluted with additional DI water to 1 Liter (L). The sugar solution was filtered through a 0.45 micron filter and the solution was passed through a freshly packed PEI column (25.0×1.0cm) at 4 mL/min. The solution was analyzed for Endotoxin. The endotoxin value was decreased from 25.6 Endotoxin Units per gram (EU/g) to <0.1 EU/g.

### Example 5

The purified Sugar solution was concentrated to 700-800 mg/ml and cooled to 40°-60° C. Then 2× to 3× volume of Anhydrous Alcohol was added with stirring. Once the beaker contents reached room temperature, the beaker was chilled in an 0° C. to 20° C. ice bath for two to four hours with occasional stirring The crystals formed were washed with anhydrous Alcohol and dried under vacuum at 50° C. for 4 hours. The crystals obtained using this procedure are free flowing and having particle size range from 80 micron to 500 micron.

| Sugar | Crystallization Yield (%) |
|---|---|
| Sucrose | 93 |
| Galactose | 91 |
| Trehalose dihydrate | 95 |

### Example 6

760 mg/mL Sucrose was spiked with reducing sugars such as Fructose or Dextrose, and thereafter crystallized. The solid Sucrose crystallized from the 1000 ppm spiked liquids following the typical procedure outlined above removed the reducing sugars below 200 ppm. This data suggests that the process of crystallization removes small amounts (up to 0.1%) of reducing sugars such as Fructose and Dextrose.

### Example 7

A 7.5 kilogram (kg) sugar (sucrose) was dissolved in about 25 L purified water under stirring using overhead stirrer at about stirring speed of 50 rpm to produce a solution having solid content of about 23%. The solution was stirred till clear solution was obtained. The resultant solution was then spray dried using a spray dryer having fitted with rotary atomizer having size 100 mm at a speed of about 14000 rpm. The inlet temperature of about 149-151° C., outlet temperature of about 100-108° C. and spray rate of about 5 L per hour was kept to produce spray dried sugar. A yield of about 20-25% was obtained after completion of sugar spray drying trial.

Thus while there have been described what are presently believed to be preferred embodiments of the invention, those skilled in the art will realize that changes and modifications may be made thereto without departing from the spirit of the invention, and it is intended to claim all such changes and modifications as fall within the true scope of the invention.

What is claimed is:

1. A method of making a highly pure carbohydrate composition comprising:
i) passing an aqueous carbohydrate solution through an anion exchange chromatography column to obtain a purified solution; and
ii) isolating a highly pure carbohydrate composition from said purified solution.

2. The method according to claim 1 wherein said anion exchange resins is made of polyethyleneimine (PEI).

3. The method of making a highly pure carbohydrate composition according to claim 1 wherein said isolating step includes at least one of the steps of: crystallization with an alcohol, or spray drying said purified solution.

4. The method of making a highly pure carbohydrate composition according to claim 3 wherein said crystallization step is performed with ethanol.

5. The method of making a highly pure carbohydrate composition according to claim 1 further comprising a filtration step of said aqueous carbohydrate solution before passing said aqueous carbohydrate solution through an anion exchange chromatography column to obtain a purified solution.

6. The method of making a highly pure carbohydrate composition according to claim 1 wherein said resultant composition is a colorless material free of plant derived material.

7. The method according to claim 5 wherein said filtration step comprises passing said aqueous carbohydrate solution through a filter with a pore size of about 0.4 microns to about 0.5 microns.

8. The method according to claim 1 wherein said highly pure carbohydrate composition is selected from the group of carbohydrates including sucrose, galactose, and trehalose.

9. The method according to claim 1 wherein said highly pure carbohydrate composition has endotoxin levels of less than 2.5 Endotoxin Unit per gram.

10. The method according to claim 1 wherein said highly pure carbohydrate composition has less than 5 ppb of elemental impurities such as lead.

11. The method according to claim 1 wherein said highly pure carbohydrate composition has less than 100 ppm of related carbohydrate species, preferably less than 10 ppm.

12. A highly pure carbohydrate composition comprising an aqueous carbohydrate solution having an endotoxin value of less than 1 Endotoxin Units per gram, said highly pure composition made by the method of
i) passing an aqueous carbohydrate solution through an anion exchange chromatography column to obtain a purified solution; and
ii) isolating a highly pure carbohydrate composition from said purified solution.

13. The highly pure carbohydrate composition of claim 12 wherein said aqueous carbohydrate solution has an endotoxin value of less than 0.4 Endotoxin Units per gram.

14. The highly pure carbohydrate composition of claim 12 wherein said aqueous carbohydrate solution has an endotoxin value of less than 0.3 Endotoxin Units per gram.

15. The highly pure carbohydrate composition of claim 12 wherein said aqueous carbohydrate solution has an endotoxin value of about 0.1 Endotoxin Units per gram.

16. The highly pure carbohydrate composition of claim 12 wherein said aqueous carbohydrate solution has been passed through an anion exchange chromatography column including a polyethyleneimine (PEI) chromatographic media.

17. The highly pure carbohydrate composition of claim 15 wherein said aqueous carbohydrate solution is further isolated by at least one of the steps of:
i) crystallization with an alcohol, or
ii) spray drying said purified solution.

18. The highly pure carbohydrate composition of claim 12, wherein said highly pure carbohydrate composition is selected from the group of sucrose, galactose, and trehalose.

19. The highly pure carbohydrate composition of claim 12 wherein said highly pure carbohydrate composition has less than 5 ppb of elemental impurities such as lead.

20. A formulation ingredient for a pharmaceutical composition comprising a highly pure carbohydrate composition having an endotoxin value of less than 1 Endotoxin Units per gram, said highly pure composition made by the method of
i) passing an aqueous carbohydrate solution through an anion exchange chromatography column to obtain a purified solution; and
ii) isolating a highly pure carbohydrate composition from said purified solution.
